(19) United States
(12) Patent Application Publication
(10) Pub. No.: US 2007/0055145 A1
(43) Pub. Date: Mar. 8, 2007

(54) Methods and apparatus for nuclear tomo-cardiology scanning

(76) Inventors: Deborah Ruth Zelnik, Haifa (IL); Danny Hausner, Haifa (IL)

Correspondence Address: THE SMALL PATENT LAW GROUP LLP, 611 OLIVE STREET, SUITE 1611, ST. LOUIS, MO, 63101, US

(21) Appl. No.: 11/484,187

(22) Filed: Jul. 11, 2006

Related U.S. Application Data
(60) Provisional application No. 60/710,470, filed on Aug. 23, 2005.

(51) Int. Cl. A61B 5/05

(52) U.S. Cl. 600/428; 600/425

(57) ABSTRACT

Methods and systems for nuclear tomo-cardiology scanning are provided. A patient monitoring system is provided that includes a patient monitoring unit attached to a stationary portion of an imaging scanner and a plurality of leads connected to an interface on a moving portion of the imaging scanner and communicatively coupled to the patient monitoring unit.

### CROSS REFERENCE TO RELATED APPLICATIONS

This application claims priority to and the benefit of the filing date of U.S. Provisional Application No. 60/710,470, filed on Aug. 23, 2005 entitled “METHODS AND APPARATUS FOR NUCLEAR TOMO-CARDIOLOGY SCANNING,” which is hereby incorporated by reference in its entirety.

### BACKGROUND OF THE INVENTION

This invention relates generally to medical imaging systems and, more particularly, to patient monitoring in a medical imaging system.

At least some known nuclear tomo-cardiology and computed tomography (CT) scanning procedures require attaching the patient to electrocardiogram (ECG) leads that include electrodes to sense the electrical activity due to certain physiological functions of the patient. The ECG monitor is stationary usually on a cart or attached to the wall while the patient moves on the table, for example, in and out of a bore of a gantry of a gamma camera scanner. A plurality of leads, for example, 2 to 12 leads connect the ECG monitor to the patient. Electrical wires connect the ECG monitor to a control computer of the gamma camera scanner.

During a scan, the ECG leads are coupled between the stationary ECG and the patient's chest and move with the patient into and out of the bore of the scanner gantry. The leads may tangle in the table and can also cut off and break. This adds time and cost to the scanning process. Also, the cable to the control computer can cause people to trip as they move within the examination room.

### BRIEF DESCRIPTION OF THE INVENTION

In one embodiment, a patient monitoring system is provided that includes a patient monitoring unit attached to a stationary portion of an imaging scanner and a plurality of leads connected to an interface on a moving portion of the imaging scanner and communicatively coupled to the patient monitoring unit.

In another embodiment, a medical imaging system is provided that includes a scanner configured to image an object and having a movable table on a stationary table frame. The medical imaging system further includes a plurality of patient leads connected to the movable table.

In yet another embodiment, a method for providing patient monitoring in connection with imaging is provided. The method includes providing a patient monitoring unit in a fixed orientation and allowing movement of a plurality of patient leads connected to the patient monitoring unit. The plurality of leads move along with a movable table of an imaging system.

### BRIEF DESCRIPTION OF THE DRAWINGS

FIG. 1 is a perspective view of an exemplary imaging system constructed in accordance with an embodiment of the invention.

FIG. 2 is a schematic block diagram of the imaging system shown in FIG. 1.

FIG. 3 is a perspective view of an exemplary imaging scanner constructed in accordance with an embodiment of the invention that may be used with the system shown in FIG. 1 and illustrating an ECG lead configuration.

FIG. 4 is perspective view of a portion of an exemplary imaging scanner constructed in accordance with an embodiment of the invention that may be used with the system shown in FIG. 1 and illustrating ECG leads coming out of a patient table.

### DETAILED DESCRIPTION OF THE INVENTION

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

Although various embodiments of the invention may be described herein relative to a particular imaging system, for example, a nuclear medicine system, other medical imaging modalities, such as computed tomography (CT), single positron emission tomography (SPECT), positron emission tomography (PET), nuclear magnetic resonance imaging (MRI), static X-ray imaging, dynamic (Fluoroscopy) X-ray imaging, and multimodality combinations thereof may also benefit form the methods described herein and the use of the present invention is contemplated with respect to these modalities. The various embodiments may be implemented in connection different imaging modalities that are operated in a “gated mode” wherein data acquisition is synchronized with a heart pulse. For example, PET and MRI may be used while electroencephalogram (EEG) data is acquired.

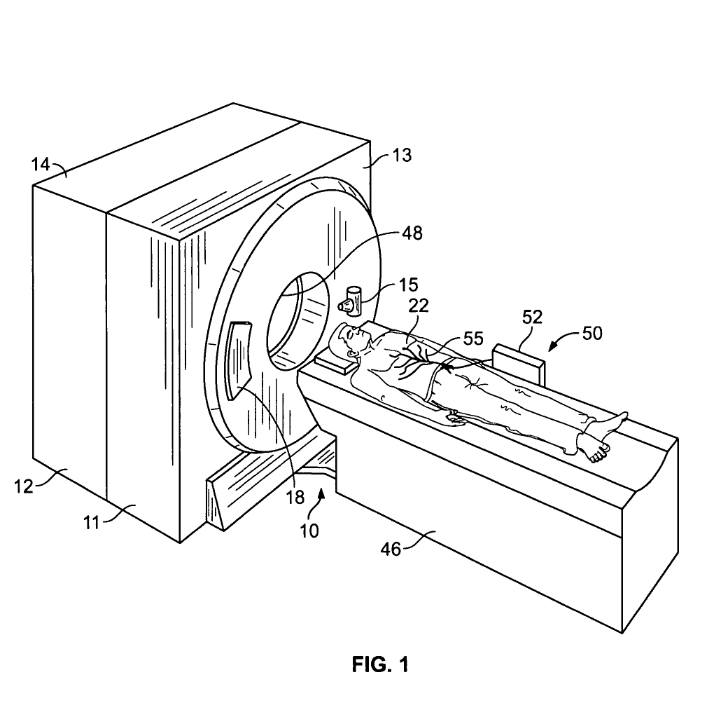

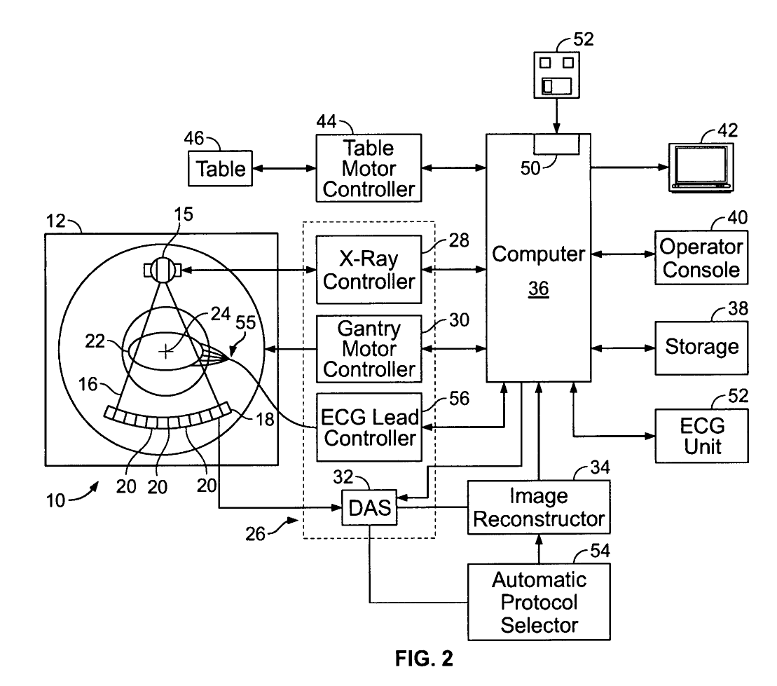

FIG. 1 is a perspective view of an exemplary imaging system 10 constructed in accordance with various embodiments of the invention. FIG. 2 is a schematic block diagram of the imaging system 10 (shown in FIG. 1). In the exemplary embodiment, the imaging system 10 is a multi-modal imaging system and includes a first modality unit 11 and a second modality unit 12. The modality units 11 and 12 enable the system 10 to scan an object, for example, a patient, in a first modality using the first modality unit 11 and to scan the object in a second modality using the second modality unit 12. The system 10 allows for multiple scans in different modalities to facilitate an increased diagnostic capability over single modality systems. In one embodiment, the multi-modal imaging system 10 is a Computed Tomography/Positron Emission Tomography (CT/PET) imaging system 10. The CT/PET system 10 includes a first gantry 13 associated with the first modality unit 11 and a second gantry 14 associated with the second modality unit 12. In alternative embodiments, modalities other than CT and PET may be employed with the imaging system 10. The gantry 13, in an embodiment, includes the first modality unit 11 that has an x-ray source 15 that projects a beam of x-rays 16 toward a detector array 18 on the opposite side of the gantry 13. The detector array 18 is formed by a plurality of detector rows (not shown) including a plurality of detector elements 20 that together sense the projected x-rays that pass through an object, such as a patient 22. Each detector element 20 produces an electrical signal that represents the intensity of an impinging x-ray beam and allows estimation of the attenuation of the beam as the beam passes through the object or patient 22.

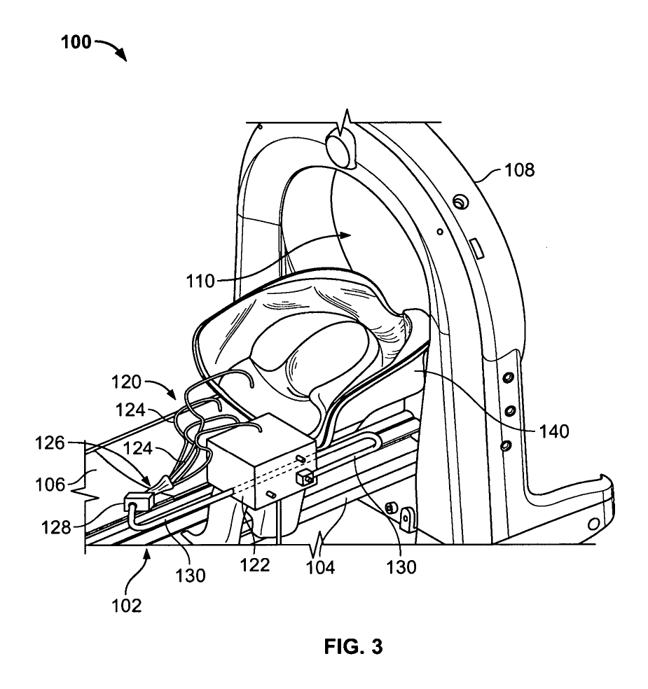

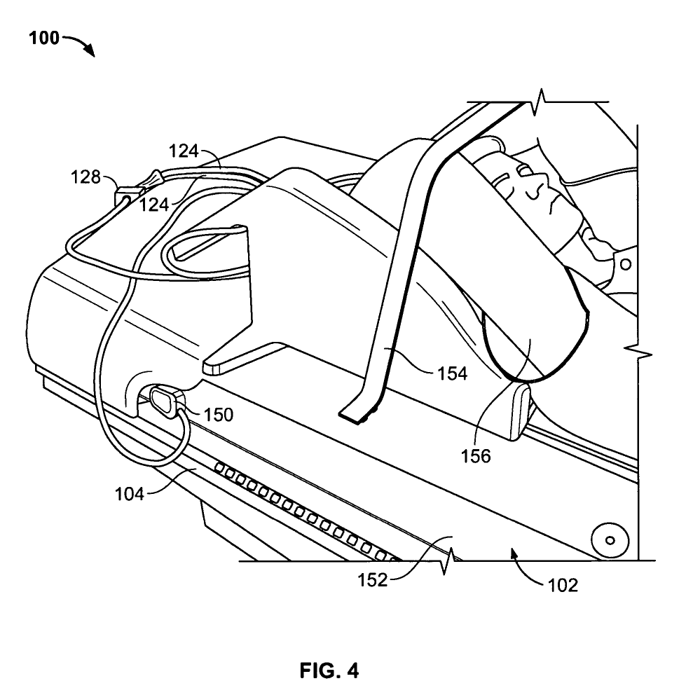

In the various embodiments, the system 10 also includes a patient monitoring system with a patient monitoring unit, for example, an electrocardiogram (ECG) system 50, exemplary embodiments of which are described in more detail below in connection with FIGS. 3 and 4. The ECG system 50 generally includes an ECG monitor 52 and patient leads 54 for attaching to a patient during an exam to monitor ECG activity with the ECG monitor 52.

In other embodiments, the system 10 includes only a single gantry having a first rotor configured to carry the first modality system and a second rotor configured to carry the second modality system. In various other embodiments the system 10 includes only one modality, such as CT, X-ray, NM or PET.

During a scan to acquire x-ray projection data the gantry 13 and the components mounted thereon rotate about an examination axis 24. FIG. 2 shows only a single row of detector elements 20 (i.e., a detector row). However, the detector array 18 may be configured as a multislice detector array having a plurality of parallel detector rows of detector elements 20 such that projection data corresponding to a plurality of slices can be acquired simultaneously during a scan. To acquire emission data, the gantry 14 rotates one or more gamma cameras (not shown) about the examination axis 24. The gantry 14 may be configured for continuous rotation during an imaging scan and/or for intermittent rotation between imaging frames.

The rotation of the gantries 13 and 14, and the operation of the x-ray source 15 are controlled by a control mechanism 26 of the system 10 (e.g., CT/PET system). The control mechanism 26 includes an x-ray controller 28 that provides power and timing signals to the x-ray source 15 and a gantry motor controller 30 that controls the rotational speed and position of the gantry 13 and the gantry 14. A data acquisition system (DAS) 32 of the control mechanism 26 samples data from the detector elements 20 and the gamma cameras and conditions the data for subsequent processing. An image reconstructor 34 receives sampled and digitized x-ray data and emission data from the DAS 32 and performs high-speed image reconstruction. The reconstructed image is transmitted as an input to a computer 36 that stores the image in a storage device 38.

The computer 36 also receives commands and scanning parameters from an operator via console 40 that has an input device, such as, a keyboard. An associated display 42 allows the operator to observe the reconstructed image and other data from the computer 36. Operator supplied commands and parameters are used by the computer 36 to provide control signals and information to the DAS 32, the x-ray controller 28 and the gantry motor controller 30. In addition, the computer 36 operates a table motor controller 44 that controls a motorized table 46 to position the patient 22 in the gantry 13 and 14. Specifically, the table 46 moves portions of the patient 22 through the gantry opening 48.

The computer 36 further receives commands from the ECG unit 52 via a user input, for example, provided as part of the ECG unit 52. The ECG unit 52 controls an ECG lead controller 56 that is communicatively coupled to the patient leads 54. The ECG lead controller 56 is configured to control the communication of information between the ECG unit 52 and the patient leads 54. It should be noted that the ECG system 50 (shown in FIG. 1) may be a separate unit or integrated with the system 10. Further, the ECG unit 52 also may be connected directly to the ECG lead controller 56 or connected directly to the patient leads 54.

In one embodiment, the computer 36 includes a read/write device 50, for example, a floppy disk drive, CD-ROM drive, DVD drive, magnetic optical disk (MOD) device, or any other digital device including a network connecting device such as an Ethernet device for reading instructions and/or data from a computer-readable medium 52, such as a floppy disk, a CD-ROM, a DVD or an other digital source such as a network or the Internet, as well as yet to be developed digital means. In another embodiment, the computer 36 executes instructions stored in firmware (not shown). The computer 36 is programmed to perform functions as described herein, and as used herein, the term computer is not limited to integrated circuits referred to in the art as computers, but broadly refers to computers, processors, microcontrollers, microcomputers, programmable logic controllers, application specific integrated circuits, and other programmable circuits, and these terms are used interchangeably herein. The system 10 may also includes a plurality of other detectors, for example, PET detectors (not shown) including a plurality of detector elements. The PET detectors and the detector array 18 both detect radiation and are both referred to herein as radiation detectors.

An automatic protocol selector 54 is communicatively coupled to the DAS 32 and the image reconstructor 34 to transmit settings and parameters for use by the DAS 32 and the image reconstructor 34 during a scan and/or image reconstruction and image review. Although the automatic protocol selector 54 is illustrated as a separate component, it should be understood that that functions performed by the automatic protocol selector 54 may be incorporated into functions performed by, for example the computer 36. Accordingly, the automatic protocol selector 54 may be embodied in a software code segment executing on a multifunctional processor or may embodied in a combination of hardware and software.

Additionally, although described in a medical setting, it is contemplated that the embodiments of the invention may be implemented in connection with other imaging systems including industrial CT systems such as, for example, but not limited to, a baggage scanning CT system typically used in a transportation center such as, for example, but not limited to, an airport or a rail station, non-destructive testing systems, etc.

FIG. 3 is a perspective view of an exemplary imaging scanner, for example, a nuclear tomo-cardiology scanner 100 that may be used with the imaging system (shown in FIG. 1). Various embodiments of the invention provide a patient monitoring system with a patient monitoring unit, such as an ECG system for connection to the scanner 100. In an exemplary embodiment, the scanner 100 includes a motorized table 102. The motorized table 102 includes a stationary table frame 104 and a movable table portion 106. A gantry 108 with a plurality of detector elements (not shown) includes a bore 110 therethrough. The movable table portion 106 is supported on the stationary table frame 104 and may include movable members (e.g., wheels or sliding elements) configured to allow movement of the movable table portion 106, for example, laterally in and out of the gantry 108 through the bore 108, with the stationary table frame 104 remaining in a fixed position. It should be noted that in an alternate embodiment the stationary table frame 104 also may be configured to provide upward and downward movement of the movable table portion 106.

In an exemplary embodiment, the patient monitoring system is an ECG system 120 constructed in accordance with various embodiment of the invention for use with an imaging scanner, for example, the nuclear tomo-cardiology scanner 100, and includes a patient monitoring unit, such as an integrated electrocardiogram (ECG) unit 122 (e.g., an ECG monitor) coupled to a side of the motorized table 102, and more particularly, to the stationary table frame 104. The ECG unit 122 may be any type of ECG system as is known, such as a GE Medical EKG system available from GE Healthcare. The ECG system 120 also includes patient leads 124 (e.g., two to twelve leads) configured to attach to a patient during a scan. More particularly, the patient leads 124 include electrode ends that attach to the patient during a scan and are configured to acquire electrical signals relative to the electrical activity of a bodily function of the patient, for example, the activity associated with the patient heart.

The patient leads 124 also include a connection end 126 for coupling to the movable table portion 106, and more particularly, an interface, for example, a junction box 128 (e.g., a combiner or splitter unit) provided on the movable table portion 106. This connection to the movable table portion 106 allows the patient leads 124 and the junction box 128 to move with the movable table portion 106, for example, in and out of the bore 110 during a scanning operation. In another exemplary embodiment, the ECG unit 122 is removably connected to a shelf (not shown) that is connected to stationary table frame 104. For example, the ECG unit 122 may include a local display (not shown) as is known and that allows viewing of scanning information provided on the supported stationary ECG unit 122.

The ECG system 120 includes a retractor cable 130 (a portion of which is shown in phantom dashed lines) that connects the junction box 128 on the movable table portion 106 to the ECG unit 122. The retractor cable 130 is an intermediate cable configured, for example, to follow the motion of the movable table portion 106, for example, move along with the movable table portion 106 when the movable table portion 106 moves in and out of the bore 110. In an exemplary embodiment, a recoiling system (not shown) may be provided in any known manner such that the retractor cable 130 extends and retracts as the movable table portion 106 moves in and out of the bore 110. In another exemplary embodiment, the retractor cable 130 may include a protective sheath fabricated to bend in a preferred direction and to restrict bending in other directions. The protective sheath includes therein the electrical cables and other links to provide information, control signals, power, etc. between the ECG leads 124 and the ECG unit 122. Information and data received by the ECG unit 122 also may be communicated to, for example, a workstation, such as a camera workstation located separate from the scanner 100.

The scanner may include a patient head and arm support 140 coupled to an end (e.g., front or leading end) of the movable table portion 106. The head and arm support 140 is configured to substantially fix the patient head and arms in a repeatable and comfortable position during a scan and may be constructed as described in U.S. Patent Application Ser. No. 60/710,709, filed on Aug. 23, 2005, entitled “METHODS AND APPARATUS FOR ERGONOMIC ARM AND HEAD SUPPORT,” assigned to the assignee of the present invention, the entire disclosure of which is hereby incorporated by reference herein. The head and arm support 140 may be removably connected (e.g., snap fit) to the movable table portion 106 in any known manner. The head and arm support 140 generally includes a plurality of depressions to maintain the position of the arms of a patient around and above the head of the patient during a scanning operation. Thus, the head and arm support 140 is configured to substantially fix the patient head and arms in a repeatable and comfortable position during a scan.

The various embodiments described herein may be modified. For example, in an alternate embodiment of the scanner 100 shown in FIG. 4, the ECG unit 122 is concealed or hidden within the motorized table 102, for example, in a base portion of the motorized table 102 or in the stationary table frame 104. In this embodiment, an interface 150 is connected to a side 152 of the movable table portion 106 to interface the retractor cable 130 (hidden within the moving portion of the motorized table 102) with the hidden ECG unit 122. The interface 150 may be configured as a junction box 128 (shown in FIG. 3). Additionally, one or more restraints 154 may be provided to immobilize a portion of a patient 156, for example, a head of the patient 156.

Although various embodiments of the invention are described herein relative to a particular imaging system, for example, a nuclear medicine system, the various embodiments may be implemented in connection with other medical imaging modalities and modified to acquire different information. For example, other patient physiological functions may be monitored using the systems described above. Also, the various

The above-described embodiments of a medical imaging system provide a cost-effective and reliable means for monitoring bodily functions of a patient during a medical imaging scan. Specifically, coupling the delicate and numerous patient leads to a junction box on the movable portion of the patient table and coupling the junction box to an ECG unit through a durable and robust cable that can be guided to follow the traversal of the patient table into and out of the gantry bore facilitates the imaging scan by reducing the probability of a cable being cut or being a nuisance.

Exemplary embodiments of medical imaging systems and apparatus are described above in detail. The medical imaging system components illustrated are not limited to the specific embodiments described herein, but rather, components of each system may be utilized independently and separately from other components described herein. For example, the medical imaging system components described above may also be used in combination with different medical imaging system components.

A technical effect of the various embodiments of the systems and methods described herein include facilitating operation of the medical imaging system by reducing the likelihood that electrical leads will become tangled or destroyed during a scan.

While the invention has been described in terms of various specific embodiments, those skilled in the art will recognize that the various embodiments of the invention can be practiced with modification within the spirit and scope of the claims.

What is claimed is:

1. A patient monitoring system comprising:
a patient monitoring unit attached to a stationary portion of an imaging scanner; and
a plurality of leads connected to an interface on a moving portion of the imaging scanner and communicatively coupled to the patient monitoring unit.

2. A patient monitoring system in accordance with claim 1 wherein the stationary portion comprises a stationary table frame.

3. A patient monitoring system in accordance with claim 1 wherein the moving portion comprises a movable table portion.

4. A patient monitoring system in accordance with claim 1 wherein the interface comprises a junction box connecting the plurality of leads to a cable from the patient monitoring unit.

5. A patient monitoring system in accordance with claim 4 wherein the cable is configured to provide retractable operation.

6. A patient monitoring system in accordance with claim 1 further comprising a shelf mounted to the stationary portion and supporting the patient monitoring unit in a stationary position.

7. A patient monitoring system in accordance with claim 1 wherein the patient monitoring unit is positioned concealed within a motorized table of the imaging scanner and further comprising another interface on the motorized table and connecting the plurality of leads via a cable to the concealed patient monitoring unit.

8. A patient monitoring system in accordance with claim 1 wherein the imaging scanner comprises a nuclear tomo-cardiology scanner and the patient monitoring unit comprises one of an electrocardiogram (ECG) unit and an electroencephalogram (EEG) unit.

9. A patient monitoring system in accordance with claim 1 further comprising a cable in a protective sheath connecting the interface to the patient monitoring unit.

10. A medical imaging system comprising:
a scanner configured to image an object and having a movable table on a stationary table frame; and
a plurality of patient leads connected to the movable table.

11. A medical imaging system in accordance with claim 10 further comprising an interface on the movable table connecting the plurality of patient leads to one of an electrocardiogram (ECG) monitor and an electroencephalogram (EEG) monitor.

12. A medical imaging system in accordance with claim 11 wherein the monitor is connected to the stationary table frame such that the monitor is stationary during a scan and the plurality of patient leads move with the movable table during the scan.

13. A medical imaging system in accordance with claim 11 further comprising a shelf connected to the stationary table frame and wherein the monitor is supported on the shelf.

14. A medical imaging system in accordance with claim 11 wherein the monitor is concealed within the stationary table frame.

15. A medical imaging system in accordance with claim 10 further comprising a cable connecting the plurality of patient leads to an ECG unit, the cable moving with the movable table.

16. A medical imaging system in accordance with claim 15 further comprising a protective sheath surrounding the cable and configured to bend in a preferred direction and resist bending in other directions.

17. A medical imaging system in accordance with claim 15 wherein the cable is configured to move in a plurality of directions relative to the movable table.

18. A medical imaging system in accordance with claim 10 wherein the scanner comprises a nuclear tomo-cardiology scanner.

19. A method for providing patient monitoring in connection with imaging, said method comprising:
providing a patient monitoring unit in a fixed orientation; and
allowing movement of a plurality of patient leads connected to the patient monitoring unit, the plurality of patient leads moving along with a movable table of an imaging system.

20. A method in accordance with claim 19 wherein the patient monitoring unit is mounted to a stationary portion supporting the movable table.
